(19) United States
(12) Patent Application Publication
(10) Pub. No.: US 2006/0120969 A1
(43) Pub. Date: Jun. 8, 2006

(54) Medical product for inhalation containing glucagon-like peptide-1 (GLP-1)

(75) Inventors: Thomas Nilsson, Mariefred (SE); Sven Calander, Straengnaes (SE); Alf Niemi, Straengnaes (SE); Claes Friberg, Akers Styckebruk (SE); Lars Kax, Nykvarn (SE); Mattias Myrman, Stockholm (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: Microdrug AG, Hergiswil NW (CH)

(21) Appl. No.: 11/049,696

(22) Filed: Feb. 4, 2005

(30) Foreign Application Priority Data
Dec. 3, 2004 (SE) 0402976-5

(51) Int. Cl. A61K 38/26; A61K 9/14; A61L 9/04

(52) U.S. Cl. 424/46; 514/12

(57) ABSTRACT

A medical product containing an accurately metered dose of a GLP-1 medicament intended for pulmonary inhalation put into a moisture-tight, high barrier seal container. The medical product optionally also contains a dose of insulin. The container is preferably adapted for application into a dry powder inhaler. The dose loaded in the container is intended for a prolonged delivery by inhalation to the deep lung where the active ingredients are absorbed into the system. Optionally the medical product also may comprise at least one biologically acceptable excipient.

### REFERENCE TO PRIOR APPLICATIONS

This application claims priority from Swedish patent application SE0402976-5 filed Dec. 3, 2004, incorporated herein by reference.

### FIELD OF THE INVENTION

The present invention relates in general to a medical product containing a metered medication dose of a glucagon-like peptide-1 (GLP-1) in dry powder form and more particularly to a metered GLP-1 dose enclosed in a moisture-tight container adapted for use in a dry powder inhaler, capable of systemic dose delivery.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Administering systemically acting drugs directly to the lungs of a patient by means of an inhaler is an effective, quick and user-friendly method of drug delivery, especially compared to administration by injections. A number of different inhaler devices have been developed in order to deliver drugs to the lung, e.g. pressurized aerosol inhalers (pMDIs), nebulizers and dry powder inhalers (DPIs).

The lung is an appealing site for systemic delivery of drugs as it offers a large surface area (about 100 m²) for the absorption of the molecules across a thin epithelium, thus having a potential for rapid drug absorption. Pulmonary delivery of drugs has the potential of attaining a high, rapid systemic drug concentration often without the need of penetration enhancers. The feasibility of this route of administration for a particular drug depends on, for example, dose size and extent and ease of systemic absorption through the alveols of the particular drug. Important factors for the deposition of inhaled particles in the lung are inspiration/expiration pattern and the particle aerodynamic size distribution. The aerodynamic particle size (AD) of the drug particles is important if an acceptable deposition of the drug within the lung is to be obtained. In order for a particle to reach into the deep lung the aerodynamic particle size should typically be between 1 and 3 μm. Larger particle sizes will easily stick in the mouth and throat and will be swallowed. Smaller particles, on the other hand, may not have time to settle and may follow the expiration air out again. Thus, it is important to keep the aerodynamic particle size distribution of the dose within tight limits to ensure that a high percentage of the dose is actually deposited where it will be most effective. The aerodynamic diameter (AD) of a particle is defined as the diameter of a spherical particle having a density of 1 g/cm³ that has the same inertial properties in air as the particle of interest. If primary particles form aggregates, the aggregates will aerodynamically behave like one big particle in air.

GLP-1 is synthesized in intestinal endocrine cells in two principal major molecular forms, as GLP-1(7-36) amide and GLP-1(7-37). These molecules are secreted in response to nutrient ingestion and play multiple roles in metabolic homeostasis following nutrient absorption. Biological activities include stimulation of glucose-dependent insulin secretion and insulin biosynthesis, inhibition of glucagon secretion and gastric emptying and inhibition of food intake. The substance plays an important role in lowering blood glucose levels in diabetics by stimulating the beta-cells in pancreas to produce insulin. A very interesting effect of GLP-1 is that it normalizes blood glucose levels in response to hyperglycemic conditions without the risk of ending up in a hypoglycemic condition. Also, GLP-1 helps control satiety and food intake. The substance therefore constitutes an interesting pharmacological drug, particularly so for treatment of diabetes, preferably in combination with insulin or even as an alternative to a regimen of insulin. See European Patent EP 0 762 890 B 1.

GLP-1 is a relatively small peptide molecule with a great potential for inhalation therapy. Fortunately, provided that the GLP-1 powder formulation is constituted of particles of the right size to sediment in the deep lung after inhalation, GLP-1 has been shown to be soluble in the fluid layer in the deep lung and dissolve, thereby ensuring rapid absorption from the lung into the system before enzymatic inactivation sets in. See for instance U.S. Pat. No. 6,720,407.

From a stability point of view, a solid formulation stored under dry conditions is normally the best choice. In the solid state, these molecules are normally relatively stable in the absence of moisture or elevated temperatures. GLP-1 and analogues or derivatives thereof in dry powder form are more or less sensitive to moisture depending on the powder formulation.

GLP-1 may be administered to humans by any available route, but oral or parenteral administration may be the most common methods. Frequent injections, necessary for the management of a disease, is of course not an ideal method of drug delivery and often leads to a low patient compliance as they infringe on the freedom of the patient as well as because of psychological factors. Tablets or capsules given orally have a fairly long onset and may suffer from low efficacy because of metabolic degradation of the GLP-1 substance before it passes into the system. Pulmonary absorption is therefore an interesting alternative, which potentially offers a fast onset, less degradation and higher efficacy. Tests have shown that users, given a choice, prefer inhalation of medicaments to self-injection.

Hence, there is a demand for precisely matched, therapeutic pulmonary dosages of GLP-1 type medicaments, especially in dry powder formulations and optionally in combination with insulin, and high efficacy devices for delivering dosages to the system by inhalation.

### DETAILED DESCRIPTION OF THE PREFERRED EMBODIMENTS

The present invention discloses a medical product comprising an accurately metered dose of a GLP-1 medicament intended for pulmonary inhalation put into a moisture-tight, high barrier seal container. The medical product optionally also comprising a dose of insulin. The container is preferably adapted for application in a dry powder inhaler. The dose loaded into the container is preferably adapted and intended for a prolonged delivery by inhalation to the deep lung where the active ingredients are absorbed into the system. Optionally the medical product also comprises at least one biologically acceptable excipient.

The present invention includes a medicament containing as active ingredient a therapeutically effective amount of least one GLP-1 substance selected for example from any one of the following sequences, and physiologically acceptable salts thereof:

R1-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-R2

wherein R1—is selected from a group consisting of His-, (Lys)₆-His- and Asn-(Glu)₅-His-, and -R2 is selected from a group consisting of -Pro-Pro-Ser-(Lys)₆, -Ser and -Ser-(Lys)₆.

The GLP-1 medicament preferably exists in dry powder form suitable for administration by inhalation, optionally comprising at least one biologically acceptable excipient.

In a further aspect of the present invention the GLP-1 agent or medicament is combined with a suitable insulin dry powder formulation, whereby the medication combination of a GLP-1 dosage and an insulin dosage are administered by inhalation as dry powder(s) in a regimen of therapeutically effective dosages to a user in need thereof. Particularly, the combined dosages may be administered together as a single formulation, a single preparation, a mixture of powders or administered separately as part-doses but in a single inhalation or administered separately by separate inhalation of each part-dose.

In another aspect of the present invention a method for the treatment of diabetes type 1 and type 2 is disclosed, which comprises administering by inhalation to a host in need of such treatment, effective amounts of at least one GLP-1, and/or physiologically acceptable salt(s) thereof, and/or solvate(s) thereof, and optionally in combination with inhalation of effective amounts of insulin.

Of course, it is within the invention for a doctor to prescribe the invention materials to patients in need thereof.

### BRIEF DESCRIPTION OF THE DRAWINGS

The invention, together with further objects and advantages thereof, may best be understood by referring to the following detailed description taken together with the accompanying drawings, in which:

FIG. 1 illustrates in a timing diagram the concentration of GLP-1 in the system of a diabetic user after inhalation of a small dose in connection with meals during a day, compared to a big dose once a day;

FIG. 2 illustrates in a timing diagram the concentration of insulin in the system of a diabetic user after inhalation of a combined dose of GLP-1 and insulin in connection with meals during a day;

FIG. 3 illustrates in two timing diagrams a typical inhalation and dose delivery of the medical product according to the present invention;

FIG. 4 illustrates in perspective, top and side views a first embodiment of a medical product comprising a dose loaded into a high barrier seal container;

FIG. 5 illustrates in top and side views a second embodiment of a medical product comprising a dose loaded into a high barrier seal container, here illustrated in an opened state;

FIG. 6 illustrates in a top view a third embodiment of several similar medical products comprising differently sized doses loaded into identical high barrier seal containers;

FIG. 7 illustrates in top and side views a second embodiment of a medical product comprising a combined dose loaded into two separate high barrier seal containers, adapted for insertion together into a DPI.

The present invention includes an improved medical product comprising an accurately metered medication dose of at least one GLP-1 in dry powder form, the dose being enclosed in a container presenting a high barrier seal. The active GLP-1 agent may optionally include at least one biologically acceptable excipient. The dose is preferably adapted and intended for systemic absorption by pulmonary inhalation in a prolonged dose delivery using a dry powder inhaler device. The present invention makes it possible to deliver an exact, high efficacy powder dosage of GLP-1 to the system of a user via the deep lung.

The pharmacological actions of glucagon-like peptide-1 or analogues and derivatives thereof, in this document generically called GLP-1, include stimulation of insulin release, suppression of glucagon release and inhibition of gastric emptying. These actions provide one basis for this invention, where we have surprisingly found that it is possible to treat type 1 as well as type 2 diabetes by pulmonary administration of therapeutically effective amounts of GLP-1 alone or preferably in combination with a regimen of inhalable insulin.

A particular peptide agonist acting as a GLP-1 agent to be used in the present invention was described in U.S. Pat. No. 6,528,486, which hereby is included in this document in its entirety as a reference.

Another particular GLP-1 derivative, which may be used in the present invention, was described in U.S. Pat. No. 6,268,343, which hereby is included in this document in its entirety as a reference.

In a particular aspect of the present invention a GLP-1 agent is selected which is long-acting following pulmonary delivery.

In a particular aspect of the present invention a GLP-1 medicament is used as an alternative to subcutaneous insulin in the treatment of early diabetes type 2, where a regimen of the GLP-1 medicament, optionally in combination with insulin, through a pulmonary route of administration eliminates the use of subcutaneous insulin to a user.

In a further aspect of the present invention a GLP-1 medicament is used in combination with insulin in the treatment of diabetes type 1 and 2, such that a regimen of inhaled GLP-1 and insulin for instance in connection with meals three or four times per day is well adapted to the needs of a diabetic user with the objective of improving glycemic control for the user and eliminating subcutaneous insulin altogether.

In yet another particular aspect of the present invention GLP-1, administered by inhalation for pulmonary absorption into the system, optionally in combination with insulin, improves user quality of life and user compliance with a prescribed dosing regimen based on inhalation of medicaments, compared to injections or a mixture of oral administration and injections. Systemic delivery by pulmonary absorption is faster and more accurate than by subcutaneous injection, partly because of the difficulty in the latter method to control exactly where the dose will be located in the subcutaneous tissue and as a consequence the systemic concentration over time will vary considerably from one injection to the next. Furthermore, GLP-1 has a rather small therapeutic window, i.e. a too small dose will have no effect at all whereas a too big dose will often cause the user to feel sick and even vomit. The pulmonary route for GLP-1 is thus to be preferred because of fast on-set, exactness, user comfort and reduced adverse side effects.

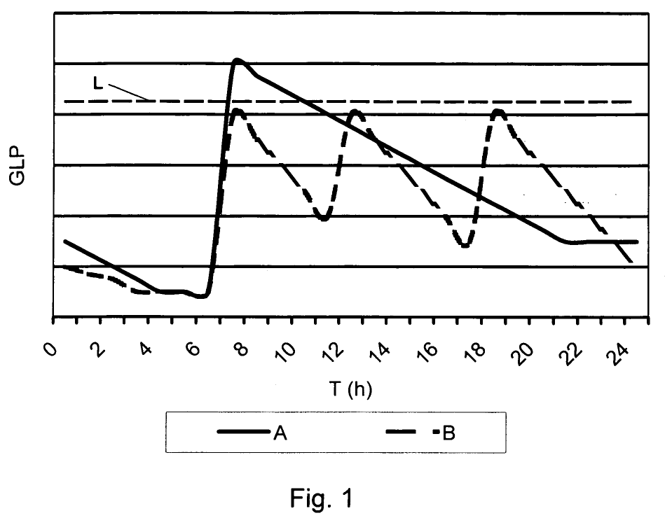

Advantageously, GLP-1 is inhaled several times daily in connection with meals, so that the GLP-1 effect on the pancreatic insulin production is not too small nor leading to too high concentration in the blood, but so that the GLP-1 concentration is kept within the optimal therapeutic window, thereby leading to a better control of glucose concentration in the blood. See FIG. 1, showing two curves, A and B over time T, representing plasma concentration of GLP-1, where curve A is the result of a single, high dose administered in the morning compared to 3 smaller doses administered in direct connection with meals during the day as in curve B. Curve A shoots over the permitted maximum level L, which causes unwanted adverse effects in a subject, such as nausea or inducing vomiting attacks. Clearly, a better way to achieving glycemic control is to administer GLP-1 in relatively small doses in connection with meals.

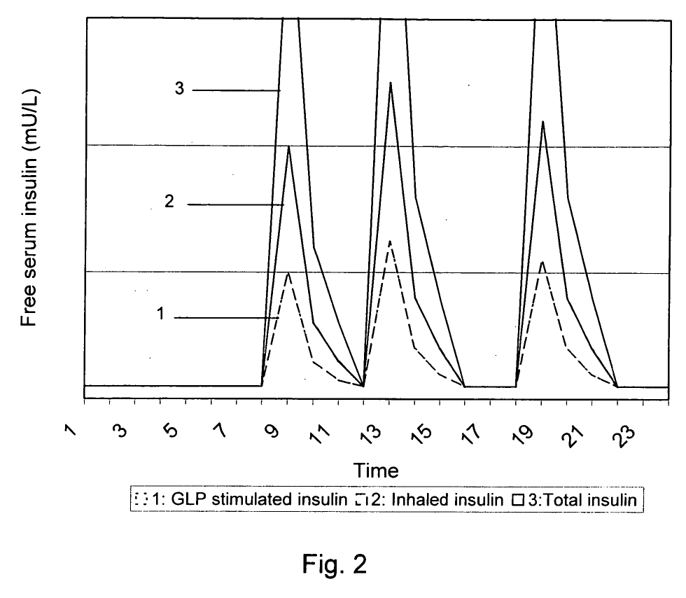

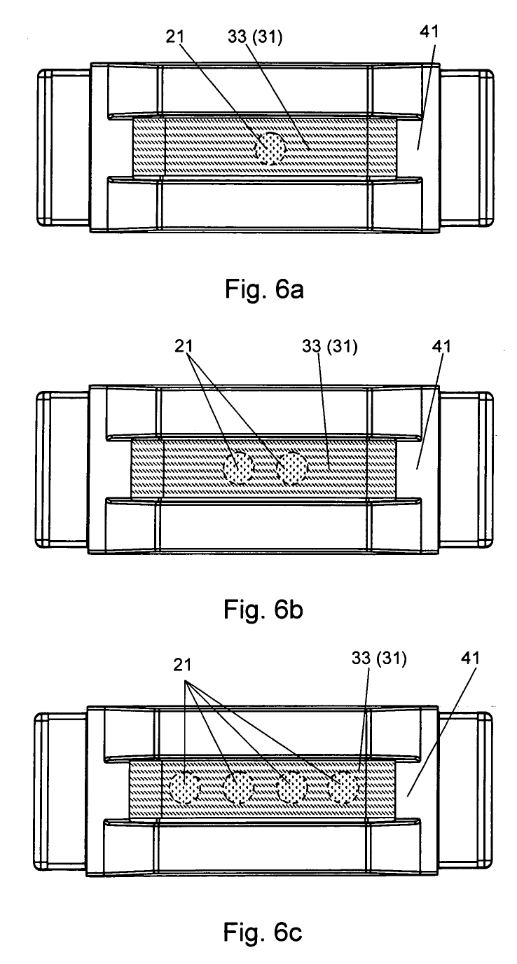

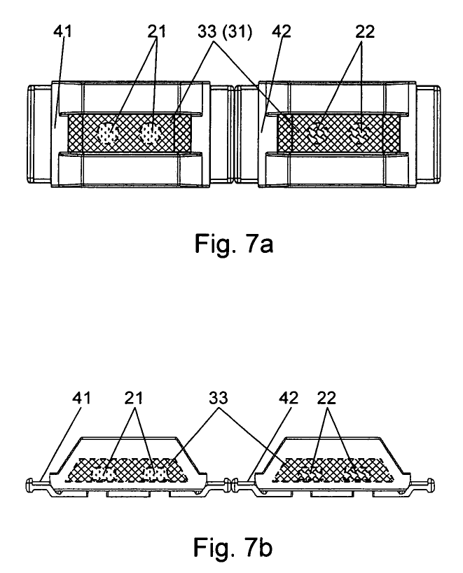

In a particular embodiment of the present invention the medical product is arranged such that an individually selected, effective dose of GLP-1 is combined with a dose of insulin, where the size of the insulin dose is selected before each administration by a diabetic user based on an estimation or actual measurement of the present level of glucose in the blood and with a regard for the imminent meal. A dry powder inhaler is thus to be loaded by the said user with a sealed container carrying a dose of GLP-1 and a similar container carrying a titratable dose of insulin, e.g. containing 10, 20, 40, 60, 100, 150 or 200 insulin units (IU). Both doses are then administered in a single inhalation. See FIGS. 7a and 7b illustrating two carriers, 41 and 42, each carrying a sealed container 33 (seal 31) containing a dose 21 of GLP-1 and a dose 22 of insulin respectively. The doses are hidden from view by the sealed container, but nevertheless indicated in the illustration for the benefit of the reader. For instance, the user has been supplied with a number of identical GLP-1 dose containers and a collection of insulin dose containers representing three different dose sizes, low, medium and high, plus empty dose containers. For example, differently sized doses 21 may be loaded into identical or similar sealed containers 33 (seal 31) and fitted to carriers 41 as illustrated in FIGS. 6a, 6b and 6c. Based on the need of the user in the course of a day, he or she decides what combination is required at each instance of administration and composes an adequate combination of GLP-1 and insulin, where the GLP-1 dose is fixed but the insulin dose is variable. The flexibility of the medical product will permit GLP-1 to stimulate the self production of insulin and only add a minimum of exogenous insulin to help control blood sugar. See FIG. 2 for graphic representations of insulin plasma concentration partly from GLP-1 stimulated endogenous insulin 1, exogenous insulin 2 and the combined insulin concentration 3 over time during a day, if a combined dose of GLP-1 and insulin is administered in connection with meals.

Self-administration of peptides, such as insulin, by subcutaneous injection is part of everyday life for many patients with diabetes. Normally, the user needs to administer insulin several times daily based on close monitoring of the glucose level. Incorrect timing of the administration or incorrect dosing may lead to hyperglycemia or hypoglycemia. Also, there are pharmacokinetical limitations when using the subcutaneous route. Absorption of insulin after a subcutaneous injection is slow. It sometimes takes up to an hour before the glucose level in the blood begins to be significantly reduced. This inherent problem with subcutaneous insulin delivery cannot be solved with a more frequent administration. In order to obtain plasma insulin concentrations that are physiologically correct over time it is advantageous to choose another route of administration, such as inhalation.

There are many advantages in combining GLP-1 and insulin in a medical product intended for administration by inhalation in the treatment of diabetes 1 and 2, such as:

Substantial reduction of insulin doses is possible

Big improvement in glycemic control

Endogenous insulin secretion is stimulated

Risk of hyperglycemia is substantially reduced

Partial or complete inhibition of insulin injections is possible

Less adverse side effects

Big improvement in user quality of life

Better user compliance

In short, a combined therapy comprising GLP-1 and insulin results in better medical status and higher quality of life for the user.

Besides diabetes 1 and 2, other important therapeutic areas of use for GLP-1 include, especially in combination with other medicaments, such as insulin, cardiovascular disorders, conditions of obesity and dyslipidaemia/lipodystrophy.

Preferably, the quality of a delivered GLP-1 dose, as well as an insulin dose, to the lung is very high in terms of fine particle fraction. As has been pointed out in the foregoing, particles preferably are 5 μm or less in aerodynamic diameter (AD) to have a reasonable chance of reaching into the deep lung when inhaled. Large particles may impact and stick in the mouth or further down in the airways before they reach the deep lung. Here, small particles may be absorbed by the alveols and delivered to the system. AD of particles should thus preferably be in a range from 0.5 to 5 μm and more preferably in a range 1 to 3 μm for a rapid and successful delivery to the system through the lung. Particles of this size sediment in the lung provided that the inhalation is deep and not too short. For maximum lung deposition, the inspiration should take place in a calm manner to decrease air speed and thereby reduce deposition by impaction in the upper respiratory tracts. Particles of AD less than 1 μm take longer to sediment and a high percentage may not sediment in the lung but follow the expiration air out instead. Small particles are more easily absorbed by the alveols, which is a further reason for the delivered dose, according to the disclosure, to present a high fine particle fraction (FPF), i.e. the fine particle dose (FPD) of the delivered dose mass should be as high as possible.

The advantages of using the inhalation power of the user to full potential in a prolonged, continuous dose delivery interval within the inhalation cycle is disclosed in our U.S. Pat. No. 6,622,723 (WO 01/34233 A1), which is hereby incorporated herein by reference in its entirety. An objective of a prolonged dose delivery is to achieve a very high level of particle de-aggregation. Prior art dry powder inhalers begin aerosolizing a dose by uncontrolled spreading of energy to the powder in the dose. In prior art the supplied energy may be of different kinds, e.g. mechanical, electric or pneumatic to name a few and combinations of different kinds are common, e.g. where the inhalation energy provided by the user is re-enforced by external sources of power to accomplish particle de-aggregation and aerosolization of the dose. But the energy thus provided is directed to the whole dose for a short time. Surprisingly, we have found that the energy thus provided becomes unevenly distributed onto and in the dose, i.e. the energy density (Ws/m3) is too low in parts of the dose for de-aggregation to come about. Thus, significant parts of the dose are aerosolized as aggregated particles and delivered as aggregates to a user, but these aggregates are too big to reach the deep lung. This is why the delivered fine particle dose (FPD) out of blisters or capsules or metered doses made available in aerosolizing chambers of a prior art inhaler is too low, representing only 20-30% of the metered dose mass.

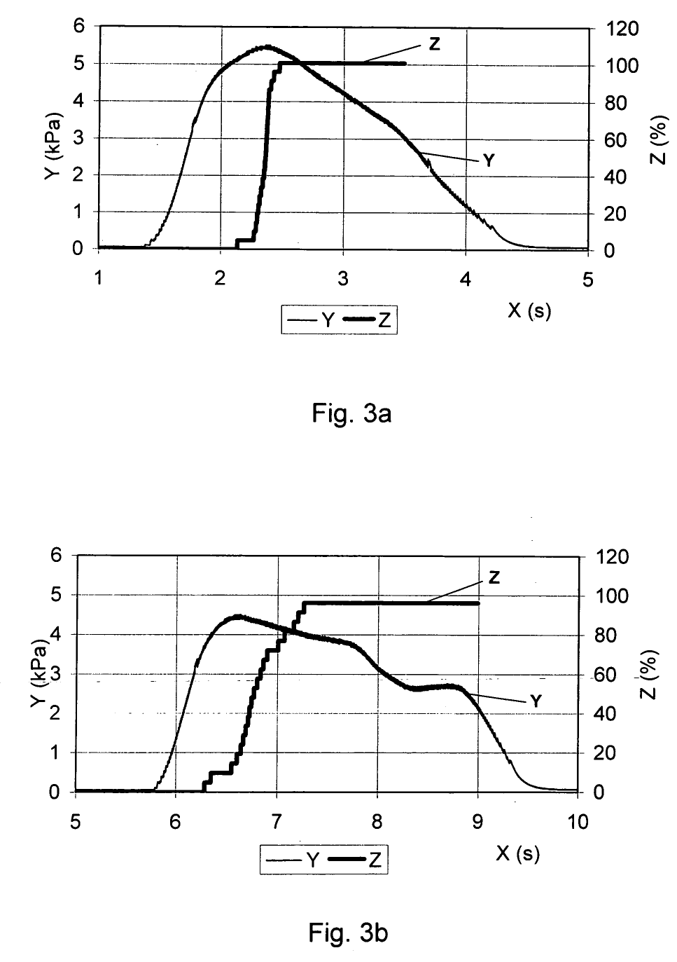

A particular solution to this problem of de-aggregation is to optimize the use of available de-aggregation energy over time by initially building up de-aggregation power quickly and then concentrate available power onto a small part of the dose only. The particles in this part-dose are completely de-aggregated and aerosolized by the high level of energy density (Ws/m3) supplied to the targeted part-dose and the de-aggregated particles are then preferably transported in the inhalation air-stream away from the rest of the dose, which remains unaffected. The available power is then shifted gradually to the rest of the dose, whereby the whole dose becomes completely de-aggregated and aerosolized over time and transported to the airways of the user. Surprisingly, we have found that if the inhalation power of a user is first allowed to build up to a certain level and then applied for a prolonged period to a single or combined dose, no other external source of power is necessary for a complete de-aggregation and aerosolization of the dose(s). A minimum level of power has been determined to be 2 kPa of suction and a normal range of suction power is 2 to 6 kPa, but typically a suction not less than 2 kPa and not greater than 4 kPa is quite satisfactory for complete de-aggregation of a single or combined dose. Preferably, the suction produces an inspiration air stream in a range 20 to 60/min and more preferably in a range 20 to 40 l/min. Arranging the medical product for a prolonged delivery in this way results in an FPD value several times higher than in prior art. Since the dose is aerosolized gradually, the dose is delivered over an interval, thereby resulting in a prolonged pulmonary dose delivery. Typically, a prolonged pulmonary dose delivery lasts from 0.1 second to 3 seconds, preferably in a range from 0.2 s to 1.5 s, depending on dose mass in the medical product and design and efficiency of the dry powder inhaler to be used. Two typical inhalation sequences are illustrated in FIGS. 3a and 3b, carried out by two subjects. Diagram curve Y represents the suction power in kPa provided by the respective subject over time X and curve Z represents dose delivery from 0 to 100% from a DPI. As can be seen, delivery of the dose does not begin until the suction is near the peak at about 4 to 5 kPa. The respective dose is fully delivered before the suction power has dropped below 4 kPa.

Surprisingly, we have found that aerosolizing the dose gradually leads to less irritation of the mucous membranes and airways of the user, with a reduced risk of coughing or choking during an inhalation. This beneficial effect is due to a reduced concentration of particles per liter inspiration air, compared to prior art combinations of dose packages and inhalers. A pro-longed delivery also uses the inhalation power provided by the user more efficiently, since the power is put to use for a long time, thereby supplying more energy to the dose for the purpose of de-aggregation. Expressed in a different way, more energy per microgram of powder is provided directly to the powder, not indirectly as is for instance the case with a dose in a capsule, which is brought to vibrate in the inhalation air stream, thereby supposedly shaking particles loose. In a further aspect of the present invention the medical product is intended for application in a single dose inhaler, which entirely relies on the power of the inhalation for de-aggregating and aerosolizing the dose, with no further external source of power necessary. See FIGS. 7a and 7b for an example of a medical product comprising a combination of GLP-1 and selectable insulin doses.

The disclosure herein is by way of example and a person of ordinary skill in the art may of course find alternative methods of energy optimization, whereby de-aggregation power of sufficient strength may be distributed evenly and efficiently onto the dose, which methods, however, are still within the scope of the present invention. See our U.S. Pat. No. 6,571,793, which is hereby incorporated herein by reference in its entirety.

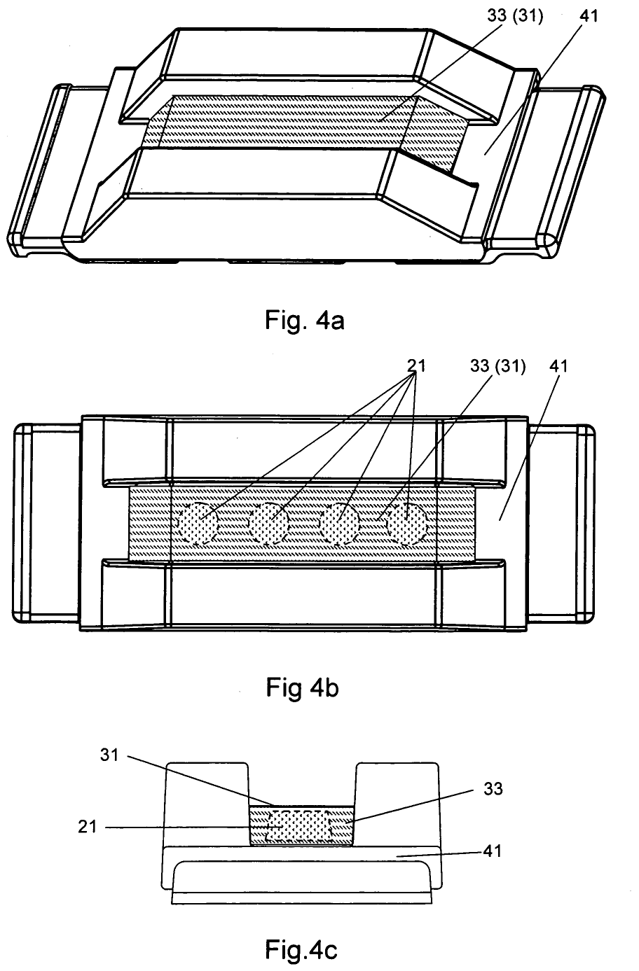

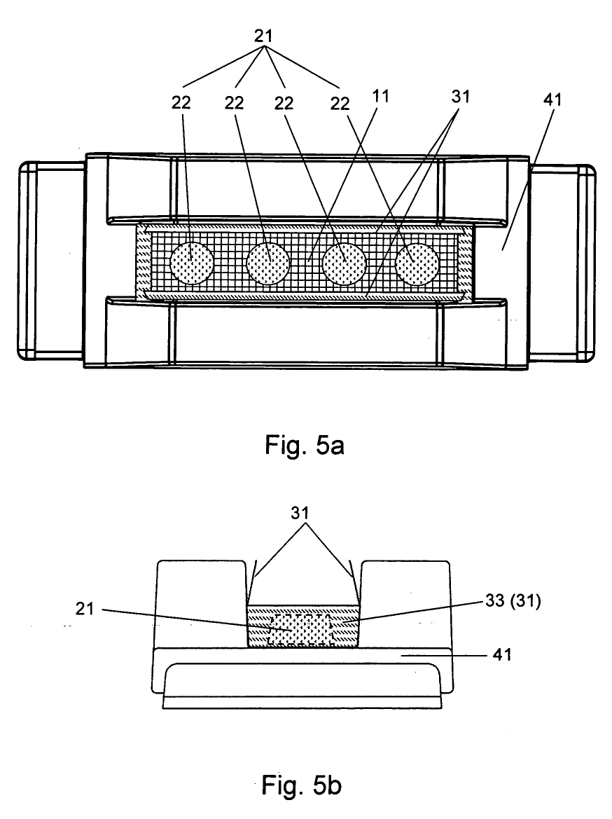

In another aspect of the invention it is important to protect a moisture-sensitive dose, such as GLP-1 or insulin, up to the very point of delivery to a user. Therefore, the medical product of the present invention should be protected by its enclosure for as long as possible after it has been made available in a dry powder inhaler. Preferably, the container of the medical product of the present invention is opened concurrently while the user performs an inhalation. In such case the time of exposing the dose powder to the atmosphere is approximately the time it takes for the delivery to take place. Any adverse effect, which depends on exposing the dose to the ambient atmosphere is thereby minimized and in practice negligible. A particular embodiment of the present invention is illustrated in FIGS. 4a, 4b and 4c. FIG. 4a shows a sealed container 33 (seal 31) put into a protective carrier 41 adapted for insertion into a dry powder inhaler. FIG. 4b shows a top view of the carrier/container and indicates depositions of dry powder making up a metered dose inside the container 33 under a seal 31, for the benefit of the reader. FIG. 4c illustrates a side view of the carrier/container in FIG. 4b. FIGS. 5a and 5b illustrate the container 33 in an opened state, where the seal 31 has been slit open and folded upwards, away from the dose 21 inside the container 33. Dose 21 is in the embodiment made up of four separate depositions 22 of dry powder. Depositions 22 may comprise same or different powders, such that the combined depositions either represent a single, metered GLP-1 dose or a combined dose of GLP-1 and insulin. A skilled person would realize that the number of depositions depends, inter alia, on the total dose mass and the relation between masses of different powders together making up a combined dose.

The fine particle fraction (FPF) of the finely divided active peptide agent, GLP-1 and optionally insulin, if present, in the metered medicament dose is preferably as high as possible, preferably having a mass median aerodynamic diameter (MMAD) below 3 μm and a particle size distribution preferably having at least 70% and more preferably more than 80% and most preferably more than 90% by mass with AD between 1 and 3 μm. After forming a metered dose, it is preferred to protect the dose from negative influences, which may otherwise detrimentally affect FPF of GLP-1 as well as insulin. Elevated temperatures have negative effects on dose stability by increasing the rate of decomposition of the active peptide agent, but moisture also constitutes a particular risk in this respect. However, moisture increases the tendency of powders to form agglomerates, which is an even greater concern, since agglomerates lower the FPF of the powder. So, in order to protect the dose according to the present invention against moisture it is preferably enclosed in a high barrier seal container, whereby the FPF of GLP-1 as well as any other component such as insulin is protected from the point of manufacture to the point of administering the respective dose, through the steps of transporting, storing, distributing and consuming.

Methods of dose forming of peptide powder formulations, e.g. GLP-1 and, e.g., insulin according to the present invention, include conventional mass, gravimetric or volumetric metering and devices and machine equipment well known to the pharmaceutical industry for filling blister packs, for example. Electrostatic forming methods may also be used, or combinations of methods mentioned. A most suitable method of depositing microgram and milligram quantities of dry powders uses electric field technology (ELFID) as disclosed in our U.S. Pat. No. 6,592,930 B2, which is hereby incorporated in this document in its entirety as a reference.

Insulin according to the present invention is defined to include insulin, insulin analogues and insulin derivatives, preferably recombinant, human insulin (“active insulin agent” or “insulin”). Methods of producing a powder formulation of a medicament intended for inhalation, such as insulin or GLP-1, generally can involve spray-drying, freeze-drying, vacuum drying or open drying, which methods result in an amorphous powder. The addition of excipients, e.g. surfactants, stabilizers and penetration enhancers is included, in the manufacturing process with the object of improving the bioavailability, speed of systemic absorption and efficacy of the medicament, for instance insulin. Methods also include making porous or hollow particles, preferably spherical in shape and geometrically bigger than 10 μm in diameter, but with AD less than 5 μm. The objectives are to get a flowable powder, which makes handling and dose forming and metering easier and yet to provide a powder, which is easy to de-aggregate when inhaled and which offers a high delivered FPD.

A particular method of preparing a dry, crystalline medicament powder before an optional mixing step, is to jet mill or micronize the ingredients of the medicament at least once and preferably twice in order to get a small mass median aerodynamic diameter (MMAD) for the finely divided powder in a range 1-3 μm with as small tails of particles outside this range as possible. The powder is then optionally mixed with one or more excipients, for example in order to dilute the potency of the active ingredient(s) to get a final powder preparation well adapted to chosen methods of metering and forming doses.

In another aspect of the present invention of combining GLP-1 and insulin in treatment of diabetes, it is advantageous to include more than one formulation of recombinant, human insulin powder in the insulin dose, e.g. in order to improve the insulin delivery into the blood circulation, such that the natural course of insulin production in a healthy person is mimicked more closely than would be possible when using only one insulin formulation. Different formulations of recombinant insulin present different absorption delays and blood concentrations over time. Therefore, a use of two or more insulin analogues in a combined dose with GLP-1 is well suited with the objective of adjusting the systemic concentration of insulin in the blood of a diabetic user over time by the combined action of the active ingredients. This treatment comes very close to bringing about the natural concentration curve in a healthy subject. When insulin is combined with administration of GLP-1, the choice of suitable insulin formulations and dosage sizes must be carefully adjusted by a person skilled in the art for best possible combination result. A typical combined therapy and dosing regimen of GLP-1 and insulin lets the diabetic user take a combined dose by inhalation just before or in connection with each meal, such as breakfast, lunch and dinner. The insulin and the GLP-1 ingredients are within minutes of inhalation absorbed into the system. The insulin helps reduce the spike of glucose following intake of food and the GLP-1 stimulates the beta-cells in pancreas to produce insulin and helps the body to keep a normal level of glucose in the blood until it is time for the next meal. In this therapy the objective of controlling a normal glucose level in the user during the day is fulfilled. Optionally, depending on the diabetic status of the user, additional doses of GLP-1 and/or insulin may be required in order to control the level of glucose during the day and night.

According to the present invention, mixing of two or more active agents into a homogenous powder mixture, optionally including one or more excipients, may be done in any order of all possible permutations, before the resulting powder mixture is used in a method of metering and forming doses. For instance, the active insulin agent may be mixed with GLP-1 first and then this mixture may be added to a mixture of excipients, if needed, but any permutation of the mixing steps may be used. The properties of the final powder mixture are decisive for the choice of mixing method, such that e.g. peptide stability is maintained, risk of particle segregation by size is eliminated and dose to dose relative standard deviation (RSD) is kept within specified limits, usually within 5%. Naturally, the ingredients must not adversely affect each other in the mixture. If there is any risk of degradation or other adverse effect in a component resulting from the mixing, then that component must not be included in the mixture, but separately administered.

In another aspect of the present invention separate dry powder dosages of GLP-1 and insulin respectively, each optionally comprising excipients, may be arranged onto a common dose carrier for insertion into an adapted inhaler and delivered to the lungs of a user, preferably in the course of a single inhalation. In a particular embodiment the separated dosages are separately enclosed onto the dose carrier in individually sealed enclosures, such as compartments, containers, capsules or blisters, known in the art. In another embodiment the separated dosages share a common enclosure onto the dose carrier. A common, sealed enclosure may be used to simplify the manufacturing process if the dosages of GLP-1 and insulin have no adverse effect on each other after deposition and sealing onto the carrier for the shelf-life of the product. The combined dosages according to the disclosure may be advantageously used in the treatment of diabetes type 1 and type 2, providing at least one of the advantages listed in the foregoing.

It is a further objective of the present invention to deliver a fine particle dose (FPD) of the at least one GLP-1 powder and optionally insulin powder if included in a combined dose, where the delivered fine particle dose amounts to at least 50% by mass, preferably at least 60% by mass, more preferably at least 70% by mass and most preferably at least 80% by mass of the active GLP-1 ingredient and optional insulin ingredient of the respective ingredients of the metered dose.

In another aspect of the invention at least one excipient is in a formulation where the MMAD of the particles is 10 μm or more, such that the at least one excipient acts as a carrier for the finely divided particles of the at least one active GLP-1 agent of the metered dose. Besides diluting the potency of the active GLP-1 ingredient(s), excipients contribute to acceptable metering and dose forming properties of the powder mixture. When the metered dose is delivered to a user by means of a dry powder inhaler device (DPI), almost all of the excipient particle mass is deposited in the mouth and upper airways, because the AD of excipient particles are generally too big to follow the inspiration air into the lung. Therefore, excipients acting as carriers and/or diluents are selected inter alia with a view to being harmless when deposited in these areas.

Suitable carrier or diluent excipients for inclusion in a GLP-1 formulation include those found among the groups of monosaccarides, disaccarides, oligo- and polysaccarides, polylactides, polyalcohols, polymers, salts or mixtures from these groups, e.g. glucose, arabinose, lactose, lactose monohydrate, lactose anhydrous [i.e., no crystalline water present in lactose molecule], saccharose, maltose, dextrane, sorbitol, mannitol, xylitol, sodium chloride, calcium carbonate. A particular excipient is lactose.

In our experience many dry powder peptides are sensitive to moisture. Thus, the moisture properties of any proposed excipient should be checked before it is chosen to be included in a formulation comprising GLP-1 and/or insulin, regardless of the intended function of the proposed excipient. If an excipient gives off much water, after dose forming, it may negatively affect the active ingredients in the dose, such that the FPD deteriorates rapidly after dose forming. Therefore, excipients are to be selected among acceptable excipients, which have good moisture properties in the sense that the excipient will not adversely affect the FPD of the active ingredients for the shelf life of the product, regardless of normal changes in ambient conditions during transportation and storage. Suitable “dry” excipients include those in the above-mentioned groups. In a particular embodiment of a GLP-1 dose, optionally also comprising insulin, lactose is selected as the preferred dry excipient and preferably lactose monohydrate. A reason for selecting lactose as excipient is its inherent property of having a low and constant water sorption isotherm. Excipients having a similar or lower sorption isotherm are also preferably considered for use, provided other required qualities are met.

The dose size depends on the type of disorder and the selected GLP-1 agent for adequate therapy, but naturally age, weight, gender and severity of the medical condition of the subject undergoing therapy are important factors. According to the present invention, a delivered fine particle dose (FPD) of the active ingredient administered by inhalation herein is not limited, and may generally be in a range from 100 μg to 25 mg, although preferably in a range from 0.5 to 25 mg. Normally, of course, a physician prescribes a proper dose size. Depending on the potency of the active substance, such as GLP-1 and human insulin agents, the active dose mass is optionally diluted by adding a pharmacologically acceptable excipient to suit a particular method of dose forming and to achieve a pre-metered dose in the inhaler exceeding 100 μg. Besides acting as a diluent, the excipient may optionally be selected to give desired electrical qualities to the powder mixture constituting the drug. A method for preparing a powder or powder mixture to bring about suitable electrostatic properties of the prepared powder to make the powder apt for a filling process is described in our U.S. Pat. No. 6,696,090, which is hereby incorporated in this document in its entirety by reference.

Further, the correct metered dose loaded into an inhaler for administration should be adjusted for predicted losses such as retention and more or less efficient de-aggregation of the inhaled dose. A practical lower limit for volumetric dose forming is in a range 0.5 to 1 mg. Doses smaller than an order of 1 mg can be difficult to produce and still maintaining a low relative standard deviation between doses of the order of at least 5%. Typically, though, dose masses for inhalation are in a range from 1 to 50 mg.

Ambient conditions during dose forming, metering and container sealing should be closely controlled. The ambient temperature is preferably limited to 25° C. maximum and relative humidity preferably limited to 15% Rh maximum. The powder formulation is also to be kept as dry as possible during the dose forming process. As already mentioned in the foregoing it is very important to control the electric properties of the powder and thereby controlling the use of electric charging and discharging of particles, regardless of which method of dose forming is to be used. Fine powders pick up static electric charges extremely easily, which can be advantageously used in dose forming, if the charging and discharging is under proper control. Taking the precautions mentioned ensure that only a very small, acceptable amount of water is enclosed in the dose container together with the dose and not enough to present a threat to the stability of the substance and the FPD of the metered dose. The original fine particle fraction (FPF) of the medicament dose at the packaging stage is further preserved by adopting a high barrier seal container for enclosing the metered dose. Thus, when the metered dose is later delivered by a DPI it is unaffected for the shelf life of the medical product by normal variations in ambient conditions during handling, storage and delivery.

“High barrier seal” means a dry packaging construction or material or combinations of materials. A high barrier seal is wherein it represents a high barrier against moisture and that the seal itself is ‘dry’, i.e. it cannot give off measurable amounts of water to the load of powder. A high barrier seal may for instance be made up of one or more layers of materials, i.e. technical polymers, aluminum or other metals, glass, silicon oxides etc that together constitutes the high barrier seal. If the high barrier seal is a foil, a 50 μm PCTFE/PVC pharmaceutical foil is a preferred high barrier foil if a two-week in-use stability for a moisture sensitive medicament shall be achieved. For longer in-use stabilities metal foils like aluminum foils from Alcan Singen can be used.

A “high barrier seal container” is a mechanical construction made to harbor and enclose a moisture sensitive dose of e.g. GLP-1 or insulin or a dose combination or a mixture thereof. The high barrier container is preferbly built using high barrier seals constituting the enclosing, i.e. walls of the container. A high barrier seal container can be made in many different shapes, e.g. completely or partly spherical, cylindrical, box-like etc. However, the volume of the container is preferably not bigger than necessary for loading and enclosing a metered dose or dose combination, thereby minimizing the amount of moisture enclosed in the atmosphere. Another requirement is that the container is designed to facilitate opening thereof, preferably in a way that makes the enclosed dose accessible for direct aerosolization and entrainment of the powder in inspiration air during an inhalation. The time the dose is exposed to ambient air is thereby minimized.

A high barrier seal container to be loaded with a dose of GLP-1 medicament is preferably made from aluminum foils of high barrier seal quality and approved to be in direct contact with pharmaceutical products. Aluminum foils that work properly in these aspects generally contain technical polymers laminated with aluminum foil to give the foil the correct mechanical properties to avoid cracking of the aluminum during forming. Sealing of the formed containers is normally done by using a thinner cover foil of pure aluminum or laminated aluminum and polymer. The container and cover foils are then sealed together using at least one of several possible methods, for instance:

using a heat sealing lacquer, through pressure and heat;

using heat and pressure to fuse the materials together;

ultrasonic welding of the materials in contact.

The sealed, dry, high barrier container of the present invention that is directly loaded with a peptide dose may be in the form of a blister and it may e.g. comprise a flat dose bed or a formed cavity in aluminum foil or a molded cavity in a polymer material, using a high barrier seal foil against ingress of moisture, e.g. of aluminum or a combination of aluminum and polymer materials. The sealed, dry, high barrier container may form a part of an inhaler device or it may form a part of a separate item intended for insertion into an inhaler device for administration of pre-metered doses. A particular embodiment of a sealed high barrier container used in an adapted DPI has the following data:

Container internal volume: 100 mm3

Effective diffusion area: 46 mm2

Diffusion constant: 0.044 g/m2 for 24 hours at 23° C. and differential Rh=50%

In a further aspect of the present invention the medical product comprises at least one GLP-1 agent and at least one active insulin agent in a combined metered dose, optionally including at least one biologically acceptable excipient, loaded and sealed into a high barrier seal container. A GLP-1 dosage and an insulin dosage, which together constitute a combined dose, may be sharing the same high barrier seal container or the dosages may be separated into separate high barrier seal containers. Methods of producing the combined dose are known in the art and include spray-drying, lyophilizing, vacuum drying, open drying, jet milling and mixing. Each ingredient may be produced as separate formulations or may be introduced into a selected process producing a combined formulation of the ingredients, if safe with regard to chemical and biological stability and toxicology. It is further possible, according to the disclosure herein, to make the resulting formulation(s) as powder, optionally powder mixtures, of finely divided particles, or large-sized porous particles. The high barrier seal container of the medical product is thus protecting the combined dose from ingress of moisture and other foreign matter, thereby preserving the FPD of the combined peptide medicament. Deterioration of the FPD is further protected by enclosing only an insignificant quantity of moisture inside the container together with the dose by keeping the humidity in the atmosphere during dose metering and forming to a sufficiently low level, and optionally by choosing the biologically acceptable excipient with as low sorption coefficient as possible. For instance, the humidity in the atmosphere where the powder is handled immediately prior to metering and forming should be kept below 15% Rh and preferably below 10% Rh, more preferably below 5% Rh and most preferably below 1% Rh. The disclosed medical product warrants that the quality of the delivered dose is high and intact over the full shelf life period and the in-use period of the product.

In Figures, 4, 5, 6 and 7 reference numbers 11-41 of the drawings same numbers indicate like elements throughout the different embodiments of the medical product, presented here as non-limiting examples.

As used herein, the phrases “selected from the group consisting of,” “chosen from,” and the like include mixtures of the specified materials. All references, patents, applications, tests, standards, documents, publications, brochures, texts, articles, instructions, etc. mentioned herein are incorporated herein by reference. Where a numerical limit or range is stated, the endpoints are included. Also, all values and sub-ranges within a numerical limit or range are specifically included as if explicitly written out.

In the context of this document all references to ratios, including ratios given as percentage numbers, are related to mass, if not explicitly said to be otherwise.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A medical product comprising a metered, dry powder, active medicament dose of at least one glucagon-like peptide-1 (GLP-1) selected from the group consisting of SEQ ID NOs: 1-9 and mixtures thereof,
loaded in a sealed container, said medical product adapted for a prolonged pulmonary delivery by inhalation from a dry powder inhaler, wherein
the medical product further comprises at least one active insulin agent, the insulin agent comprising at least one peptide of recombinant, human insulin;
and wherein the dose of the medical product is arranged to be aerosolized and entrained into inspiration air directly from the container when opened and delivered by an inhaler, and arranged to be aerosolized exclusively by the inhalation power of a user for the prolonged pulmonary delivery, whereby more than 50% by mass of active agents present in the medical product leave the inhaler as fine particle doses (FPD).

2. The medical product according to claim 1, wherein
the product is adapted such that the prolonged pulmonary delivery of a dose of the medical product takes place in a period of not less than 0.2 s and not more than 1.5 s.

3. The medical product according to claim 1, wherein
the product is adapted such that the required inhalation power for de-aggregating and aerosolizing a dose of the medical product is not less than 2 kPa and not more than 6 kPa of air pressure resulting in an inspiration air flow of not less than 20 l/min and not more than 60 l/min.

4. The medical product according to claim 1, wherein
the product is adapted such that more than 60% by mass of the GLP-1 peptide present in the medical product leaves the inhaler as a fine particle dose (FPD).

5. The medical product according to claim 1, wherein
the product is adapted such that a size of the insulin dose in the combined doses of GLP-1 and insulin is selected by a user individually depending on his or her need.

6. The medical product according to claim 1, wherein
a total mass of GLP-1 in said dose is 100 μg-25 mg of a total dose mass in a range from 1 mg to 50 mg.

7. The medical product according to claim 1, wherein
the metered, dry powder, active medicament dose of at least one glucagon-like peptide-1 (GLP-1) has a mass median aerodynamic diameter of 1 to 3 μm.

8. The medical product according to claim 1, further comprising at least one dry excipient selected from a group consisting of monosaccarides, disaccarides, polylactides, oligo- and polysaccarides, polyalcohols, polymers, salts, and mixtures thereof comprising particles having a diameter of 25 μm or more in an amount of more than 40% by mass based on total mass of excipient.

9. The medical product according to claim 1, wherein
the container constitutes a high barrier seal container protecting the dose from ingress of moisture and other harmful substances, whereby the integrity of the dose is fully protected for the shelf-life of the medical product.

10. A medical product comprising a metered, dry powder, medicament dose of at least one glucagon-like peptide-1 (GLP-1) active agent, the dose individually packaged in a sealed container and adapted for a prolonged pulmonary delivery by inhalation from a dry powder inhaler, wherein the GLP-1 medicament comprises at least one selected from the group consisting of SEQ ID NOs: 1-9 and mixtures thereof:
and wherein the dose of the medical product is arranged to be aerosolized and entrained into inspiration air directly from the sealed container when opened and delivered by an inhaler, and arranged to be aerosolized exclusively by the inhalation power of a user for the prolonged pulmonary delivery, whereby more than 50% by mass of an active GLP-1 ingredient present in the medical product leaves the inhaler as a fine particle dose (FPD).

11. The medical product according to claim 10, wherein
the product is adapted such that the prolonged pulmonary delivery of a dose of the medical product takes place in a period of not less than 0.2 s and not more than 1.5 s.

12. The medical product according to claim 10, wherein
the product is adapted such that the required inhalation power for de-aggregating and aerosolizing a dose of the medical product is not less than 2 kPa and not more than 6 kPa of air pressure resulting in an inspiration air flow of not less than 20 l/min and not more than 60 l/min.

13. The medical product according to claim 10, wherein
the product is adapted such that more than 60% by mass of the GLP-1 peptide present in the medical product leaves the inhaler as a fine particle dose (FPD).

14. The medical product according to claim 10, wherein
the product is adapted such that a size of the insulin dose in the combined doses of GLP-1 and insulin is selected by a user individually depending on his or her need.

15. The medical product according to claim 10, wherein
a total mass of GLP-1 in said dose is 100 μg-25 mg of a total dose mass in a range from 1 mg to 50 mg.

16. The medical product according to claim 10, wherein
the metered, dry powder, active medicament dose of at least one glucagon-like peptide-1 (GLP-1) has a mass median aerodynamic diameter of 1 to 3 μm.

17. The medical product according to claim 10, further comprising at least one dry excipient selected from a group consisting of monosaccarides, disaccarides, polylactides, oligo- and polysaccarides, polyalcohols, polymers, salts, and mixtures thereof comprising particles having a diameter of 25 μm or more in an amount of more than 40% by mass based on total mass of excipient.

18. The medical product according to claim 10, wherein
the container constitutes a high barrier seal container protecting the dose from ingress of moisture and other harmful substances, whereby the integrity of the dose is fully protected for the shelf-life of the medical product.

19. A method of administering a medical product via inhalation, comprising inhaling the medical product of claim 1 into a patient's lungs.

20. A method of administering a medical product via inhalation, comprising inhaling the medical product of claim 10 into a patient's lungs.

21. A method of treating a diabetic disorder in a human subject, comprising administering the medical product of claim 1 by inhalation to a patient in need thereof.

22. A method of treating a diabetic disorder in a human subject, comprising prescribing the medical product of claim 1 to a patient in need thereof.

23. A method of treating a diabetic disorder in a human subject, comprising administering the medical product of claim 10 by inhalation to a patient in need thereof.

24. A method of treating a diabetic disorder in a human subject, comprising prescribing the medical product of claim 10 to a patient in need thereof.


SEQUENCE LISTING (9 sequences)

SEQ ID NO: 1 - protein, 44 aa, Artificial Sequence
  Synthetic Peptide
HGEGTFTSDLSKQMEEEAVRLFIEWLKNGGPSSGAPPSKKKKKK

SEQ ID NO: 2 - protein, 36 aa, Artificial Sequence
  Synthetic Peptide
HGEGTFTSDLSKQMEEEAVRLFIEWLKNGGPSSGAS

SEQ ID NO: 3 - protein, 42 aa, Artificial Sequence
  Synthetic Peptide
HGEGTFTSDLSKQMEEEAVRLFIEWLKNGGPSSGASKKKKKK

SEQ ID NO: 4 - protein, 50 aa, Artificial Sequence
  Synthetic Peptide
KKKKKKHGEGTFTSDLSKQMEEEAVRLFIEWLKNGGPSSGAPPSKKKKKK

SEQ ID NO: 5 - protein, 42 aa, Artificial Sequence
  Synthetic Peptide
KKKKKKHGEGTFTSDLSKQMEEEAVRLFIEWLKNGGPSSGAS

SEQ ID NO: 6 - protein, 48 aa, Artificial Sequence
  Synthetic Peptide
KKKKKKHGEGTFTSDLSKQMEEEAVRLFIEWLKNGGPSSGASKKKKKK

SEQ ID NO: 7 - protein, 50 aa, Artificial Sequence
  Synthetic Peptide
NEEEEEHGEGTFTSDLSKQMEEEAVRLFIEWLKNGGPSSGAPPSKKKKKK

SEQ ID NO: 8 - protein, 42 aa, Artificial Sequence
  Synthetic Peptide
NEEEEEHGEGTFTSDLSKQMEEEAVRLFIEWLKNGGPSSGAS

SEQ ID NO: 9 - protein, 48 aa, Artificial Sequence
  Synthetic Peptide
NEEEEEHGEGTFTSDLSKQMEEEAVRLFIEWLKNGGPSSGASKKKKKK